(19) United States
(12) Patent Application Publication
(10) Pub. No.: US 2006/0094024 A1
(43) Pub. Date: May 4, 2006

(54) Electrochemical arrays

(76) Inventors: Michael C. Pirrung, Cardiff, CA (US); H. Holden Thorp, Carrboro, NC (US)

Correspondence Address: MYERS BIGEL SIBLEY & SAJOVEC, PO BOX 37428, RALEIGH, NC, 27627, US

(21) Appl. No.: 10/978,789

(22) Filed: Nov. 1, 2004

(51) Int. Cl. C12Q 1/68; G01N 1/28; C12M 1/34

(52) U.S. Cl. 435/6; 435/287.2; 427/2.11; 205/777

(57) ABSTRACT

The present invention relates to methods for synthesizing nucleic acids. The invention also relates to the production of an array of nucleic acids as well as methods for making such an array. Electrochemical methods may be used to both fabricate and interrogate the nucleic acid arrays.

### FIELD OF THE INVENTION

The present invention generally relates to fabricating and interrogating arrays. More specifically the present invention relates to electrochemical methods used to both fabricate and interrogate nucleic acid arrays.

### BACKGROUND OF THE INVENTION

Hybridization microarray technology has evolved to become an important tool in large scale genomics studies. Briefly, microarrays derive their name from the small size of the analysis sites typically arranged in a two-dimensional matrix of probe elements on the surface of a supporting substrate. The range of microarray samples is varied. Generally, each probe element comprises numerous identical oligonucleotide molecules. These probes are fixed to the substrate surface and may hybridize with complementary oligonucleotide “targets” from a sample. Typically, a label (e.g., fluorescent molecule) is either attached to the target prior to the hybridization step, or to the probe/target complex subsequent to hybridization. The microarrays are then observed for the presence of detectable labels (fluorescence imaging). The presence of a label in the area encompassing a particular probe element indicates that a sequence complementary to the characteristic sequence of that element was in the analyte.

Current microarray production techniques continue to evolve to permit larger arrays and the increasingly tight packaging of probe elements such that a single substrate array might allow the detection and quantation of 100,000 or more target sequences at once. A number of microarray data acquisition technologies and methodologies are known in the art, the purpose of each of which is to acquire a collection of data reflecting the pattern of hybridization on the microarray substrate.

### SUMMARY OF THE PRESENT INVENTION

The present invention relates to methods for synthesizing a nucleic acid comprising providing a first nucleic acid having a 3′-terminal and a 5′-protecting group; covalently coupling said 3′-terminal of the first nucleic acid to an electrode; and cleaving said 5′-protecting group from said first nucleic acid by passing a current therethrough under reaction conditions in which said first nucleic acid remains covalently coupled to said electrode to provide a deprotected nucleic acid.

The present invention also relates to an array of nucleic acids comprising a microelectronic substrate having at least a first surface; an oligonucleotide capture probe immobilized on said first surface; and a plurality of different oligonucleotides attached to the first surface of the substrate at a density exceeding 1000 different nucleic acids/cm², wherein each of the different nucleic acids is attached to the surface of the substrate in a different known location, and has a different determinable sequence; wherein each of said different nucleic acids has a different binding detection electrode operatively associated therewith.

Additionally the present invention includes methods of fabricating an array of nucleic acids. These include methods of providing a plurality of nucleotides with a 3′-terminal and a 5′-protecting group. The 3′-terminal of one of the nucleotides can be covalently coupled to a first surface of a microelectronic substrate. The nucleotides selected can have different predetermined sequences and can be attached at different localized areas having a width of less than 100 microns on the first surface of a microelectronic substrate. The methods can also include cleaving the 5′-protecting group from a first nucleotide attached to the microelectronic substrate by passing a current therethrough under reaction conditions in which said first nucleotide remains covalently coupled to said electrode to provide a deprotected nucleotides. Additionally, a 3′-terminal of a subsequent nucleotide can be covalently coupled to the first nucleic acid. Furthermore the first nucleotide coupled to the microelectronic substrate can have a different binding detection electrode operatively associated therewith.

### BRIEF DESCRIPTION OF THE DRAWINGS

FIG. 1 illustrates methods of synthesizing a nucleic acid on a substrate.

FIG. 2 demonstrates a method of electrochemically deprotecting the 5′ group on a nucleic acid.

FIG. 3 illustrates a flow chart diagram depicting embodiments of the present invention for electrochemically controlled DNA synthesis.

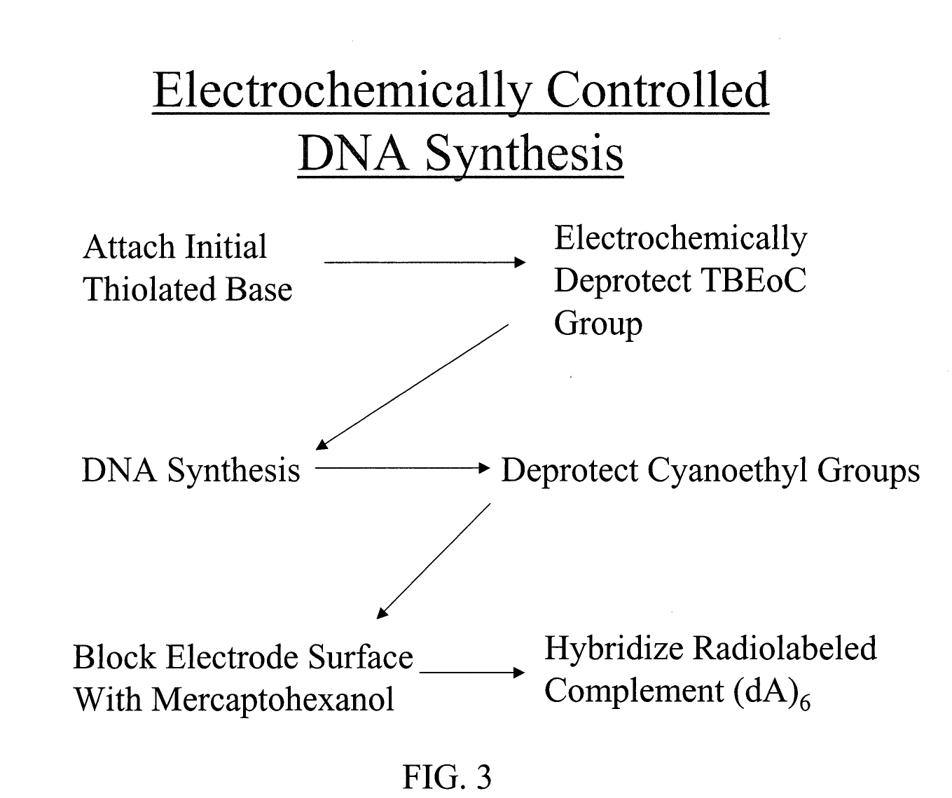

FIG. 4 depicts electrochemically controlled DNA synthesis and the amount of nucleic acids produced by the synthesis.

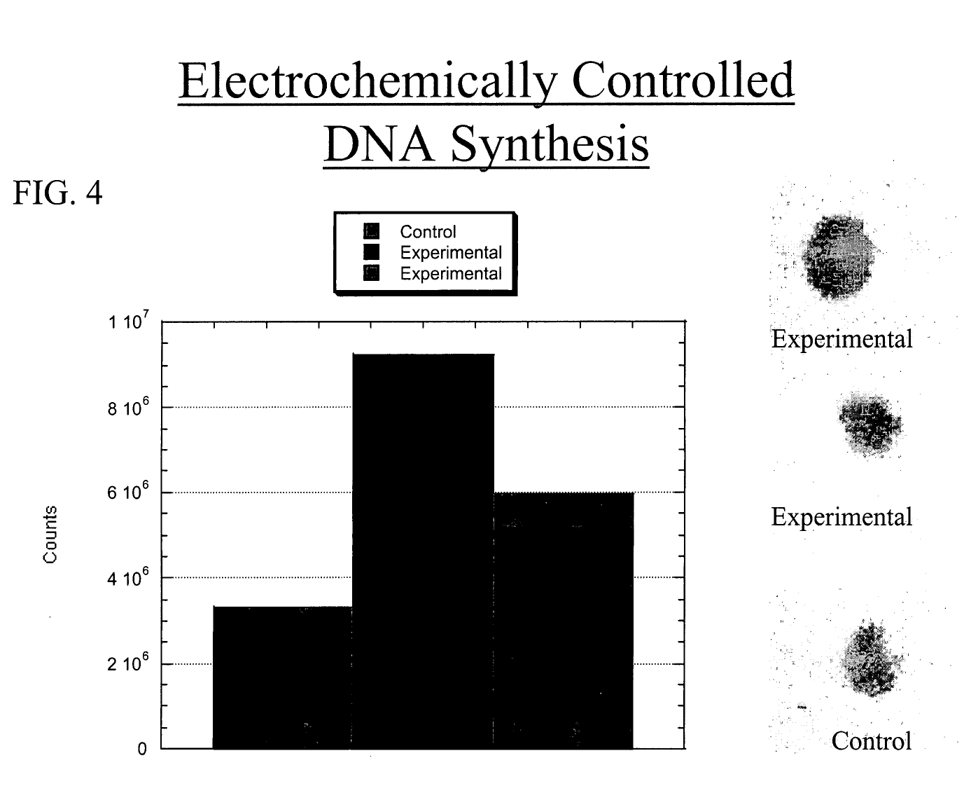

FIG. 5 depicts an assembly with an electrode and a linker attached to the thiol.

### DETAILED DESCRIPTION OF THE PREFERRED EMBODIMENTS

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs.

All publications, patent applications, patents and other references cited herein are incorporated by reference in their entireties for the teachings relevant to the sentence and/or paragraph in which the reference is presented.

The term “nucleic acid” as used herein refers to any nucleic acid, including both DNA and RNA. Nucleic acids of the present invention are typically polynucleic acids; that is, polymers of individual nucleotides that are covalently joined by 3′, 5′ phosphodiester bonds.

The term “nucleotide” refers to a building block of DNA and RNA, consisting of a nitrogenous base, a five-carbon sugar, and a phosphate group.

The term “electrode” refers to any medium capable of transporting charge (e.g. electrons). Preferred electrodes are metals (e.g., gold, aluminum), non-metals (e.g., conductive oxides, carbides, sulfides, selinides, tellurides, phosphides, and arsenides such as cadmium sulfide, cadmium telluride, tungsten diselinide, gallium arsenide, gallium phosphide, etc.), and conductive organic molecules. The electrodes can be manufactured to virtually any 2-dimensional or 3-dimensional shape. The term “electrode” can also include a conductive substrate having a working surface formed thereon; and/or a polymer layer connected to the working surface. The polymer layer is one that binds the nucleic acid (e.g., by hydrophobic interaction or any other suitable binding technique) and can be porous to the transition metal complex (i.e., the transition metal complex can migrate to the nucleic acid bound to the polymer).

The conductive substrate may be a metallic substrate or a non-metallic substrate, including semiconductor substrates (e.g., gold, glassy carbon, indium-doped tin oxide, etc.). The conductive substrate may take any physical form, such as an elongated probe having a working surface formed on one end thereof, or a flat sheet having the working surface formed on one side thereof.

The polymer layer may be connected to the working surface by any suitable means, such as by clamping the polymer layer to the working surface, evaporation of a solution of the polymer onto the electrode (i.e., evaporative deposition), or electropolymerization. Suitable polymers include polystyrene and poly (ethylene terephthalate). The thickness of the polymer layer is not critical, but can be from 100 Angström (D) to 1, 10, or even 100 microns. The polymer layer is preferably oxidized, and is then preferably modified by binding a coupling agent such as a carbodiimide thereto, in accordance with known techniques.

The term “substrate” may be a material having a rigid or semi-rigid surface. In many embodiments, at least one surface of the substrate will be substantially flat, although in some embodiments it may be desirable to physically separate synthesis regions for different polymers with, for example, wells, raised regions, etched trenches, or the like. According to other embodiments, small beads may be provided on the surface which may be released upon completion of the synthesis.

A “protective group” is a material which is bound to a monomer unit and which may be spatially removed upon selective exposure to an activator such as electromagnetic radiation. Examples of protective groups include, but are not limited to Nitroveratryloxy carbonyl, Nitrobenzyloxy carbonyl, Dimethyl dimethoxybenzyloxy carbonyl, 5-Bromo-7-nitroindolinyl, o-Hydroxy-α-methyl cinnamoyl, and 2-oxymethylene anthraquinone. The protective group can also protect the amino portion of a nucleic acid, thus forming an N-protective nucleic acid.

The term “microelectronic device” refers to a device which can be used for the electrochemical detection of a nucleic acid species in the methods described above comprises a microelectronic substrate having first and second opposing faces; a conductive electrode on the first face; and a nucleic acid capture probe immobilized on the first face adjacent the conductive electrode.

A “linker” is a molecule used to couple two different molecules, two subunits of a molecule, or a molecule to a substrate. When all are covalently linked, they form units of a single molecule. Covalent coupling can include direct covalent linkage between the molecule and the electrode, indirect covalent coupling (e.g. via a linker), direct or indirect ionic bonding between the molecule and the electrode, or other bonding (e.g. hydrophobic bonding). It may also include coupling from molecule to molecule or nucleic acid to nucleic acid. Optionally, the linker molecules may be chemically protected for storage purposes. A chemical storage protective group such as t-BOC (t-butoxycarbonyl) may be used in some embodiments. Such chemical protective groups would be chemically removed upon exposure to, for example, acidic solution and would serve to protect the surface during storage and be removed prior to polymer preparation.

Embodiments of the present invention concern methods of DNA synthesis in situ on microarrays. Embodiments of the present invention can include DNA analysis methods based on in situ electrochemical DNA probe synthesis and electrochemical complementary DNA detection. The present methods for preparation of microarrays involve either spatially directed synthesis of oligonucleotide probe (10 um resolution for photolithography) or liquid dispensing of intact oligonucleotides or cDNAs (100 um resolution).

The present invention provides synthetic strategies and devices for the creation of large scale chemical diversity. Solid-phase chemistry, photolabile protecting groups, and photolithography are brought together to achieve light-directed spatially-addressable parallel chemical synthesis in preferred embodiments.

The invention is described herein for purposes of illustration primarily with regard to the preparation of peptides and nucleotides, but could readily be applied in the preparation of other polymers. Such polymers include, for example, both linear and cyclic polymers of nucleic acids, polysaccharides, phospholipids, and peptides having either α, β, or omega-amino acids, hetero-polymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure. It will be recognized further, that illustrations herein are primarily with reference to C- to N-terminal synthesis, but the invention could readily be applied to N- to C-terminal synthesis without departing from the scope of the invention.

Embodiments of the present invention relate to the synthesis and placement materials at known locations. In one embodiment, a method and an associated apparatus may be utilized for preparing diverse chemical sequences at known locations on a single substrate surface. Embodiments of the invention may be applied, for example, in the field of preparation of oligomers, peptides, nucleic acids, oligosaccharides, phospholipids, polymers or drug congener preparations, including the ability to create sources of chemical diversity for use in screening for biological activity.

Other embodiments of the present invention can include methods for synthesizing a nucleic acid including first providing a first nucleotide having a 3′-terminal and a 5′-protecting group and then covalently coupling said 3′-terminal of the first nucleotide to an electrode. Next the 5′-protecting group is cleaved from said first nucleotide by passing a current therethrough under reaction conditions in which said first nucleotide remains covalently coupled to the electrode to provide a deprotected nucleic acid. The protective group can be cleaved from a nucleic acid by passing a current therethrough. The current may be performed at from −0 to −1000 mV, generally from −100 to −1000 mV.

In some of the embodiments of the present invention the covalently coupling can be a thiol coupling. Additional embodiments may also include providing a subsequent nucleotide having a 3′-terminal and a 5′-protecting group, covalently coupling said 3′-terminal of the subsequent nucleic acid to the deprotected nucleotide, and cleaving the 5′-protecting group from the subsequent nucleotide by passing a current therethrough under reaction conditions in which said subsequent nucleic acid remains covalently coupled to said deprotected nucleic acid and in which said deprotected nucleic acid remains covalently coupled to said electrode. Additionally these methods may be repeated by cyclically repeating these steps at least one additional time to further produce an elongated nucleic acid.

Furthermore, the embodiments of the present invention may include wherein at least one of said first nucleotide and said subsequent nucleotide is an N-protected nucleic acid and the method further comprises the step of deprotecting the at least one N-protected nucleic acid.

The electrode may be any of the noble metals including but not limited to gold, silver, platinum and palladium. Additionally, the electrode may be a metal oxide such an gold oxide, platinum oxide, silver oxide or gold platinum oxide.

The embodiments of the present invention also provide for an array of nucleic acids comprising a microelectronic substrate having at least a first surface along with a nucleic acid capture probe immobilized on said first surface, and a plurality of different nucleic acids attached to the first surface of the substrate at a density exceeding at least 1000 different nucleic acids/cm² and can be assembled into bundles as dense of 10,000,000 electrodes per cm². Each of the different nucleic acids can be attached to the surface of the microelectronic substrate in a different known location, and each can have a different determinable sequence. Additionally, each nucleic acid can have a different binding detection electrode operatively associated therewith. Each different nucleic acid can be attached to the first surface of the substrate by the 3′-terminal of a first nucleic acid. Furthermore, the contact can be electrically connected to the electrode. The array may be of any length from 1, 2, 3, 4, 5, 10, 15, 20, 25, 50, 75, 100, 150, 200, 250, 500, to well over 1,000 nucleotides in length. Each nucleic acid can be covalently coupled to the first nucleic acid.

Other embodiments of the present invention can include methods of making an array of nucleic acids comprising providing a plurality of nucleotides with a 3′-terminal and a 5′-protecting group, covalently coupling the 3′-terminal of one of the nucleotides to a first surface of a microelectronic substrate, the nucleotides having different predetermined sequences and being attached at different localized areas having a width of less than 100 microns on the first surface of a microelectronic substrate, cleaving said 5′-protecting group from a first nucleic acid attached to the microelectronic substrate by passing a current therethrough under reaction conditions in which said first nucleic acid remains covalently coupled to said electrode to provide a deprotected nucleic acid, and providing and covalently coupling a 3′-terminal of a subsequent nucleic acid having a 3′-terminal and a 5′-protecting group to the first nucleic acid.

Each of the first nucleic acid coupled to the microelectronic substrate can have a different binding detection electrode operatively associated therewith. Furthermore, an elongated nucleic acid can be produced by cyclically repeating the above steps at least one additional time. The nucleotide produced may be of any length as disclosed above.

Furthermore, the methods of making an array of nucleotides can comprise a contact electrically connected to an electrode. The 5′-protecting group can be cleaved from the first nucleic acid by passing a current therethrough under reaction conditions in which said first nucleic acid remains covalently coupled to the microelectronic substrate to provide a deprotected nucleic acid.

The invention is described herein primarily with regard to the preparation of molecules containing sequences of amino acids, but could readily be applied in the preparation of other polymers. Such polymers include, for example, both linear and cyclic polymers of nucleic acids, polysaccharides, phospholipids, and peptides having either α, β, or ω-amino acids, hetero-polymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure. In a preferred embodiment, the invention herein is used in the synthesis of peptides.

Coupling Reactions

The coupling reactions may be carried out by methods well known in the art. Theses include, but are not limited to phosphoramidite, the standard phosphodiester linkage and H-phosphonate linkage. The synthesis of DNA from β-cyanoethyl phosphoramidite monomers is currently the industry standard. With this method, high coupling efficiencies are easily attained. The absence of side reactions also confers high biological activity of the synthetic oligonucleotide. In the basic reaction cycle, a solid support, derivatized with the initial protected nucleoside, is contained in a reaction column. Reagents and solvents are pumped through the column to effect the addition of successive protected nucleotide monomers (phosphoramidites). Each addition cycle includes detritylation, activation, coupling, oxidation, and capping. Intervening wash steps remove excess reactants and by-products of reaction. After the chain elongation is complete, the oligomer must be removed from the support and fully deprotected.

H-phosphonate chemistry is of value when the intemucleotide linakge required is other than the standard phosphodiester linkage. The H-phosphonate monomers shown below are used instead of the phosphoramidite bases. Using this method, the monomer that is able to be activated is a 5′-DMT-base-protected, nucleoside 3′-hydrogen phosphonate. The presence of the H-phosphonate moiety on these monomers renders phosphate protection unnecessary. The same base protecting groups are used in phosphite triester chemistry. The H-phosphate synthesis cycle is very similar to that of the phosphoramidite method. Slight differences result from the properties of the monomers utilized. For instance, a different activating agent is used. In addition, the H-phosphonate diesteres generated by the coupling reactions are stable to the normal reaction conditions, so oxidation at every step is unnecessary. Instead, a single oxidation step can be performed at the end of the chain elongation. This single oxidation step makes it easy to produce modified DNA. For instance, if a sulfur containing compound is used as the oxidizing agent, all of the internucleotide bonds will then contain sulphur instead if oxygen attached to the phosphorous atom. H-Phosphonate synthesis uses the same supports as does the β-cyanoethyl phosphoramidite chemistry.

Protecting Groups

As discussed above, selectively removable protecting groups allow creation of well defined areas of substrate surface having differing reactivities. Preferably, the protecting groups are selectively removed from the surface by applying a specific activator, such as electromagnetic radiation of a specific wavelength and intensity. More preferably, the specific activator exposes selected areas of surface to remove the protecting groups in the exposed areas.

Protecting groups of the present invention are used in conjunction with solid phase oligomer syntheses, such as peptide syntheses using natural or unnatural amino acids, nucleotide syntheses using deoxyribonucleic and ribonucleic acids, oligosaccharide syntheses, and the like. In addition to protecting the substrate surface from unwanted reaction, the protecting groups block a reactive end of the monomer to prevent self-polymerization. For instance, attachment of a protecting group to the amino terminus of an activated amino acid, such as an N-hydroxysuccinimide-activated ester of the amino acid, prevents the amino terminus of one monomer from reacting with the activated ester portion of another during peptide synthesis. Alternatively, the protecting group may be attached to the carboxyl group of an amino acid to prevent reaction at this site. Most protecting groups can be attached to either the amino or the carboxyl group of an amino acid, and the nature of the chemical synthesis will dictate which reactive group will require a protecting group. Analogously, attachment of a protecting group to the 5′-hydroxyl group of a nucleoside during synthesis using for example, phosphate-triester coupling chemistry, prevents the 5′-hydroxyl of one nucleoside from reacting with the 3′-activated phosphate-triester of another.

Regardless of the specific use, protecting groups are employed to protect a moiety on a molecule from reacting with another reagent. Protecting groups of the present invention have the following characteristics: they prevent selected reagents from modifying the group to which they are attached; they are stable (that is, they remain attached to the molecule) to the synthesis reaction conditions; they are removable under conditions that do not adversely affect the remaining structure; and once removed, do not react appreciably with the surface or surface-bound oligomer. The selection of a suitable protecting group will depend, of course, on the chemical nature of the monomer unit and oligomer, as well as the specific reagents they are to protect against.

In some embodiments, the protecting groups can be photoactivatable. The properties and uses of photoreactive protecting compounds have been reviewed. See, McCray et al., Ann. Rev. of Biophys. and Biophys. Chem. (1989) 18: 239-270, which is incorporated herein by reference. Many, although not all, of the photoremovable protecting groups will be aromatic compounds that absorb near-UV and visible radiation. Suitable photoremovable protecting groups are described in, for example, McCray et al., Patchornik, J. Amer. Chem. Soc. (1970) 92: 6333, and Amit et al., J. Org. Chem. (1974) 39: 192, which are incorporated herein by reference. However hb based methods of making nucleic acids has physical limitation due to the size constraints.

Thus, other protecting groups include, but are not limited to benzoyl benzoate, tribromoethoxy, sulfonate esters, etc. See, Greene et al., “Protecting Groups in Organic Synthesis” (2^{nd} Edition) J. Wiley and Sons, 1991; Nucleic Acids in Chemistry and Biology, ed. G Blackburn and Gate; and Kocienski, “Protecting Groups”, Georg Thieme Verlag, 1994. This allows for prefabricated electrodes and a denser electrode filled with nucleic acids.

Substrates

Essentially, any conceivable substrate may be employed in the invention. The substrate may be biological, nonbiological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, circle, etc. The substrate is preferably flat but may take on a variety of alternative surface configurations. For example, the substrate may contain raised or depressed regions on which the synthesis takes place. The substrate and its surface preferably form a rigid support on which to carry out the reactions described herein. The substrate and its surface is also chosen to provide appropriate light-absorbing characteristics. For instance, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those of skill in is the art upon review of this disclosure.

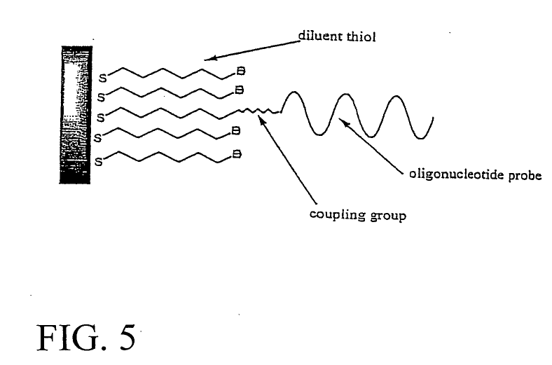

FIG. 5 illustrates a generic device comprising a gold electrode to which a linker is attached via thiol coupling. As shown in the Examples section thiol modified DNA and commercially available linkers may be synthesized via phosphoramidite chemistry. The device illustrates a gold electrode to which a mixed monolayer is attached. The mixed monolayer contains a diluent thiol with an end group that is inert. The diluent thiol should be short enough to allow for efficient electron transfer from the mediators used in the detection. The monolayer can also contain an oligonucleotide coupled to a thiol. The electrodes shown in FIG. 5 can hybridized to synthetic oligonucleotides used as targets. The target can then be detected using a catalytic electrochemistry scheme. Electrochemical DNA synthesis may then be utilized to successfully elongate the oligomer through deprotection and coupling.

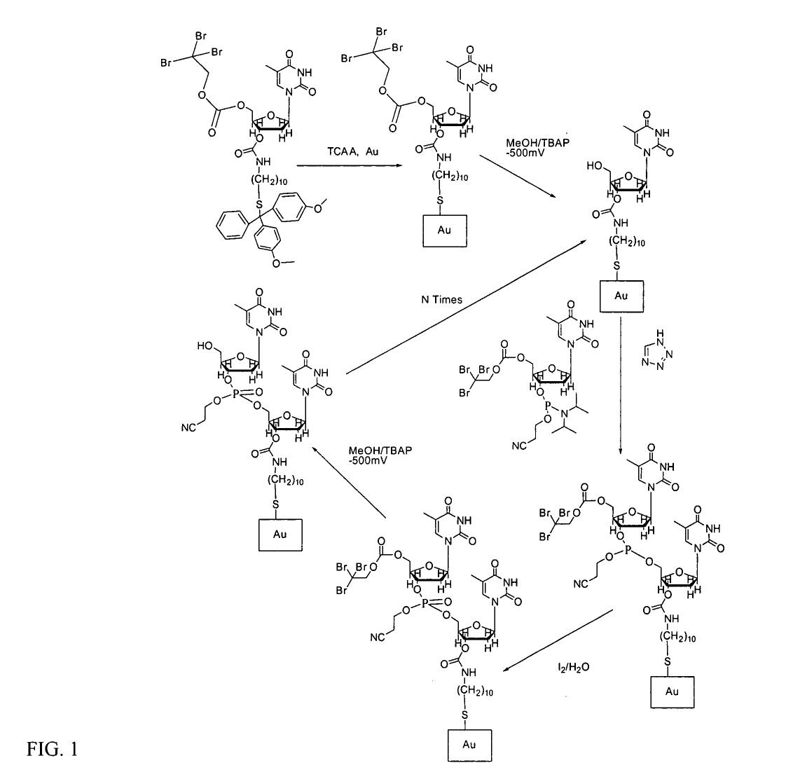

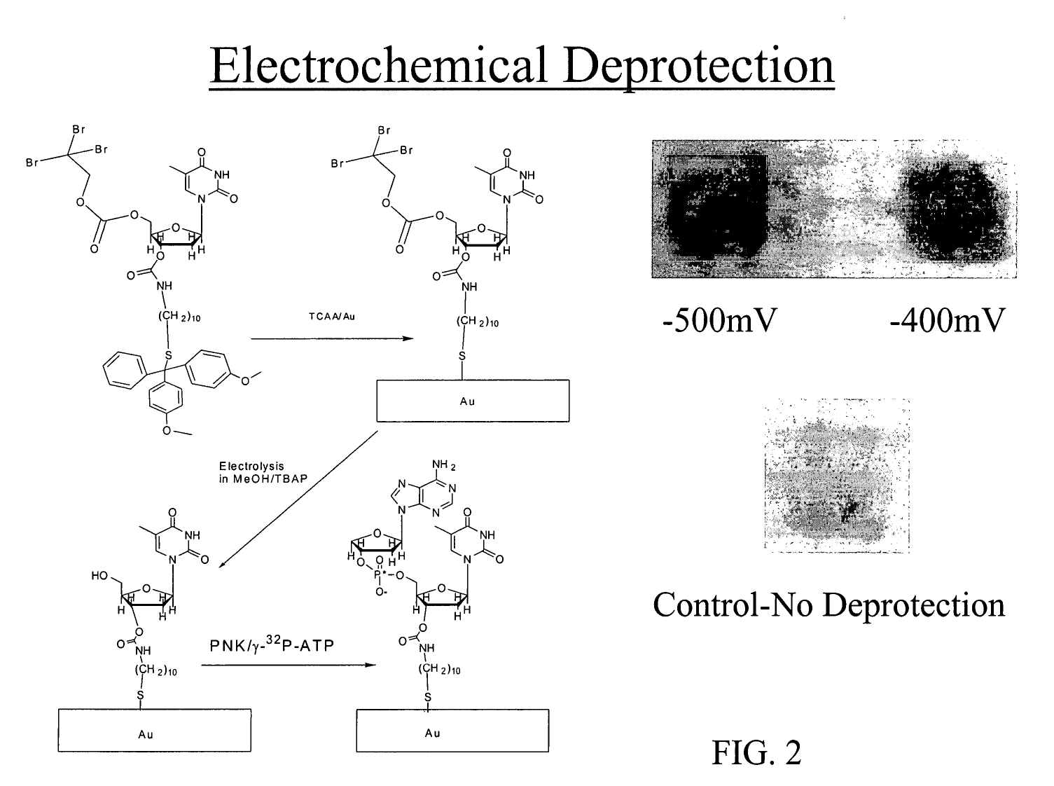

FIG. 1 illustrates a method of electrochemical deprotection. In FIG. 1 a nucleoside monomer was prepared and protected with TBEoC and identified by cyclic voltammetry conditions that permitted it to be electrolyzed at potentials that would not harm the thiol-gold self assembled monolayer. These experiments illustrate that electrolysis converts the 5′TBEoC group to the free hydroxyl. The electrolysis enables electrochemical nucleic acid synthesis. A cyclic amidine may be used with the purine bases as it is resistant to electrochemical deprotection. Additionally, the 5′TBEoC group can be converted to a thiol derivative connecting to the nucleoside by a linking chain and may be protected by a dimethoxytrityl group. These monomer units for DNA synthesis, protected at their 5′hydroxyls and derivatized at the 3′hydroxyl as the phosphoramidite may be deprotected electrochemically. FIG. 2 illustrates the deprotection at both −400 mV and at −500 mV.

The present invention is explained in greater detail in the Examples that follow. These examples are intended as illustrative of the invention and are not to be taken as limiting thereof.

### EXAMPLES

The electrodes shown in FIG. 5 are hybridized to synthetic oligonucleotides used as model targets. These synthetic oligonucleotides contain either 8-oxo-guanine or 5-aminocytosine or other modified bases. The hybridized target may be detected using a catalytic electrochemistry scheme.

Preparation of Nucleoside Derivatives.

N⁴-(N-methylpyrrolidin-2-ylidine)-2′-deoxycytidine.

2′-Deoxycytidine (0.227 g, 0.1 mmol) was co-evaporated three times with dry pyridine and dissolved in 7 mL of methanol. N-methyl-2,2-dimethoxypyrrolidine (0.174 g, 1.2 mmol) was added drop wise and the reaction mixture was stirred for 2 h with TLC monitoring for completion. The reaction mixture was concentrated and purified by flash chromatography on silica gel (3% methanol:CH₂Cl₂). The desired product was obtained as a white foam in 90% yield. UV λₘₐₓ (ε): 274 (10130), 210 (17490). ¹H NMR (DMSO, 300 MHz): δ 7.93 (d, J=7.2 Hz, 1H), 6.13 (t, J=6.6 Hz, 1H), 5.88 (d, J=6.9 Hz, 1H), 4.22-4.16 (m, 1H), 3.75-3.79 (m, 1H), 3.56-3.52 (m, 2H), 3.43 (t, J=7.2 Hz, 2H), 2.95-2.89 (m, 5H), 2.18-2.11 (m, 2H), 1.98-1.88 (m, 2H). HRMS Calcd. for C₁₄H₂₀N₄O₄: 308.1485. Found: 309.1493.

N⁶-(N-methylpyrrolidin-2-ylidine)-2′-deoxyadenosine.

2′-Deoxyadenosine (0.251 g, 0.1 mmol) was co-evaporated three times with dry pyridine and dissolved in 7 mL of methanol. N-methyl-2,2-dimethoxypyrrolidine (0.174 g, 1.2 mmol) was added drop wise and the reaction mixture was stirred for 5 h. The reaction mixture was concentrated and purified by flash chromatography on silica gel (5% methanol:CH₂Cl₂). The desired product was obtained as a white foam (0.157 g) in 51% yield. UV λₘₐₓ (ε): 313 (5940), 261 (16820), 209 (26740). ¹H NMR (DMSO, 300 MHz): δ 8.40 (s, 1H), 8.38 (s, 1H), 6.37 (t, J=6.9 Hz, 1H), 4.40-4.38 (m, 1H), 3.87-3.84 (m, 1H), 3.62-3.57 (m, 2H), 3.47 (t, J=7.2 Hz, 2H), 3.02 (s, 3H), 2.83 (t, J=7.8 Hz, 2H), 2.29-2.24 (m, 2H), 1.95-1.90 (m, 2H). HRMS Calcd. for C₁₅H₂₀N₆O₃, 332.1597. Found: 332.1593.

General Procedure for the Formation of Nucleoside 5′-O-(2,2,2-tribromoethoxycarbonyl) Derivatives.

A 0.5 M solution of 2,2,2-tribromoethylcarbonate imidazolium triflate (TBECIT) in nitromethane was prepared as follows. Methyl triflate (2.71 mL, 24 mmol) was added drop wise via syringe to a solution of 1,1′-carbonyldiimidazole (1.96 g, 12.0 mmol) in 18.5 mL of nitromethane at 0° C. The ice bath was removed after the addition and the solution was stirred for 30 min at room temperature. The solution was transferred to a flask containing 2,2,2-tribromoethanol (3.4 g, 12 mmol) which had been freshly azeotroped from benzene. The solution was stirred at room temperature for 1 h. The above reagent (20 mL) was added drop wise over 10 min to a pyridine solution (20 mL) containing 1 equiv of thymidine, which had been azeotroped twice from pyridine. The resulting solution was stirred at room temperature for 5 h and evaporated under reduced pressure. The residue was purified by chromatography.

5′-O-(2,2,2-tribromoethoxycarbonyl)-thymidine.

Elution solvent: 2:8 hexane/ethyl acetate. Yield: 30%. UV λₘₐₓ (ε): 265 (10480), 210 (13020). ¹H NMR (DMSO, 300 MHz): δ 11.27 (s, 1H), 7.46 (s, 1H), 6.18 (t, J=6.6 Hz, 1H), 4.99 (s, 2H), 4.42-4.33 (m, 2H), 4.26-4.21 (m, 1H), 3.96-3.92 (m, 1H), 2.24-2.05 (m, 2H), 1.88 (s, 3H). ¹³C NMR (DMSO, 75 MHz): δ 164.35, 153.92, 151.09, 136.58, 110.57, 84.58, 83.89, 79.56, 70.69, 69.04, 36.58, 12.97. HRMS Calcd. for C₁₃H₁₅Br₃N₂O₇: 547.8429. Found: 548.8438 (MH+).

5′-O-(2,2,2-tribromoethoxycarbonyl)-N⁴-(N-methylpyrrolidin-2-ylidine)-2′-deoxycytidine.

Elution solvent: 2% methanol/CH₂Cl₂. Yield: 25%. UV λₘₐₓ (ε): 277 (10370), 209 (16120). ¹H NMR (DMSO, 300 MHz): δ 7.93 (d, J=7.5 Hz, 1H), 6.20 (t, J=5.2 Hz, 1H), 5.94 (d, J=7.5 Hz, 1H), 5.25-5.19 (m, 2H), 5.01 (s, 2H), 4.17-4.13 (m, 1H), 3.65-3.62 (m, 1H), 3.45 (t, J=7.5 Hz, 2H), 2.97-2.92 (m, 5H), 2.30-2.20 (m, 2H), 2.00-1.89 (m, 2H). ¹³C NMR (DMSO, 100 MHz): δ 162.38, 153.24, 144.54, 94.90, 86.13, 83.43, 79.59, 61.77, 54.42, 49.14, 37.99, 36.69, 33.61, 30.76, 19.53. HRMS Calcd. for C₁₇H₂₁Br₃N₄O₆ 613.9011. Found: 614.9023.

5′-O-(2,2,2-tribromoethoxycarbonyl)-N⁶-(N-methylpyrrolidin-2-ylidine)-2′-deoxyadenosine.

Elution solvent: 3% methanol/CH₂Cl₂. Yield: 25%. UV λₘₐₓ (ε): 320 (10760), 261 (6660), 208 (20680). ¹H NMR (DMSO, 300 MHz): δ 8.71 (s, 1H), 8.67 (s, 1H), 7.76 (s, 1H), 6.45 (t, J=7.2, 1H), 4.95 (s, 2H), 4.53-4.47 (m, 1H), 4.36 (t, J=6.9, 2H), 4.12-4.01 (m, 2H), 3.65 (t, J=7.5, 2H), 3.12 (s, 3H), 3.03 (t, J=7.8, 2H), 2.90-2.81 (m, 2H), 2.08-1.98 (m, 2H). ¹³C NMR (DMSO, 100 MHz): δ 171.14, 153.08, 151.32, 144.32, 135.00, 119.94, 84.82, 79.45, 70.92, 69.00, 54.47, 49.14, 36.51, 33.53, 30.76, 19.49. HRMS Calcd. for C₁₈H₂₁Br₃N₆O₅, 637.9124, found 638.9129.

5′-O-(2,2,2-tribromoethoxycarbonyl)-3′-O-((N,N-diisopropyl)-2-cyanoethyl)phosphine)-thymidine.

To 4 mL of CH₂Cl₂ under N₂ was added 5′-O-(2,2,2-tribromoethoxycarbonyl)-thymidine (100 mg, 0.18 mmol) and triethylamine (1 mL). 2-Cyanoethyl-N,N-diisopropylchlorophosphine (70 μL, 1.5 mmol) was added and the reaction mixture was stirred for 2 h. CH₂Cl₂ (6 mL) was added, the solution was washed with NaHCO₃, the solution was evaporated and the residue subjected to column chromatography (95% yield). ³¹P NMR (DMSO, 121 MHz): δ 149.53, 149.09.

5′-O-(2,2,2-tribromoethoxycarbonyl)-3′-O-((N,N-diisopropyl)-2-cyanoethyl)phosphine)-N⁴-(N-methylpyrrolidin-2-ylidine)-2′-deoxycytidine.

70% yield. ³¹P NMR (DMSO, 121 MHz): δ 153.45, 153.72.

Triethylammonium 5′-O-(2,2,2-tribromoethoxycarbonyl)-thymidine-3′-H-phosphonate.

To a solution of 5′-O-(2,2,2-tribromoethoxycarbony)-thymidine (0.551 g, 1 mmol) in 5 mL CH₂Cl₂ was added PCl₃ (157 μL, 1.8 mmol) and imidazole (30 mg). After stirring for 1 h, the reaction mixture was quenched by addition of the mixture of water-triethylamine (1:1 v/v, 2 mL) and allowed to stand for 30 min. The solvent was evaporated and extracted with dichloromethane and followed by NaHCO₃ and dried over NaSO₄. The product was purified by chromatography (CH₂Cl₂) (80% yield). ³¹P NMR (DMSO, 121 MHz): δ 1.13 (J=682.8).

Synthesis of Linker Nucleoside

11-Mercapto-(4,4′-dimethoxytriphenylmethyl)-undecanoic acid.

11-Mercaptoundecanoic acid (0.218 g, 1 mmol) and 4,4′-dimethoxytriphenyl chloride (DMTr-Cl) (0.372 g, 1.1 mmol) were dissolved in 10 mL THF and two equiv of triethylamine was added slowly at 0° C. After stirring overnight, chromatographic purification gave the title compound in 75% yield. ¹H NMR (DMSO, 300 MHz): δ 7.40-7.25 (m, 9H), 6.80 (d, J=9.0 Hz, 4H), 3.79 (s, 6H), 2.43 (t, J=7.5 Hz, 2H), 2.14 (t, J=7.2 Hz, 2H), 1.64-1.57 (m, 4H), 1.22 (s, 12H). HRMS Calcd. for C₃₂H₄₀O₄S, 520.2647. Found, 519.2651 (M-H)⁻.

10-mercapto-(4,4′-dimethoxytriphenylmethyl)decyl-1-isocyanate.

11-Mercapto-(4,4′-dimethoxytriphenyl)-undecanoic acid (0.254 g, 0.5 mmol) and triethylamine (0.097 mL, 0.7 mmol) were dissolved in 10 mL of toluene and cooled to 0° C. under Ar. Diphenylphosphoryl azide (DPPA, 0.150 mL, 0.7 mmol) was added drop wise. After refluxing the solution for 3 h, the solvent was removed and the residue purified by chromatography (2% ethyl acetate in hexane) to give the title compound in 45% yield. ¹H NMR (DMSO, 300 MHz): δ 7.40-7.37 (m, 4H), 7.30-7.28 (m, 5H) 6.78 (d, J=9.0 Hz, 4H), 3.79 (s, 6H), 3.27 (t, J=6.6 Hz, 2H), 2.14 (t, J=7.5 Hz, 2H), 1.64-1.59 (m, 4H), 1.23 (s, 12H). HRMS Calcd. for C₃₂H₃₉NO₃S, 517.2651. Found, 516.2656 (M-H)⁻.

5′-O-(2,2,2-tribromoethoxycarbonyl)-thymidine-3′-O-(10-mercapto-(4,4′-dimethoxytriphenylmethyl)-decyl carbamate).

10-Mercapto-(4,4′-dimethoxytriphenyl)decyl-1-isocyanate (52 mg, 0.1 mmol) was dissolved in 2 mL of 1,4-dioxane and added slowly to 5′-O-(2,2,2-tribromoethoxycarbonyl)-thymidine (270 mg, 0.5 mmol) and allowed to stir for 1 h followed by reflux overnight. The solvent was removed and the product was isolated by preparative TLC in 15% yield. ¹H NMR (DMSO, 300 MHz): δ 11.35 (s, 1H), 7.50 (s, 1H), 7.29-7.27 (m, 5H), 7.17 (d, 4H, J=9.0 Hz), 6.85 (d, 4H, J=9.0 Hz), 6.18 (t, 1H, 7.5 Hz), 5.11-5.07 (m, 1H), 4.98 (s, 2H), 4.45 (d, 2H, J=4.8 Hz), 4.17-4.14 (m, 1H), 3.71 (s, 6H), 2.97-2.91 (m, 2H), 2.52-2.48 (m, 2H), 2.05 (t, 2H, J=7.8 Hz), 1.79 (s, 3H), 1.39-1.33 (m, 2H), 1.18 (s, 14H); MS (FAB+) m/e 806.0 (MH+). ¹³C NMR (DMSO, 75 MHz): δ 164.33, 161.03, 158.75, 158.27, 153.93, 151.09, 146.00, 137.50, 130.89, 129.56, 128.52, 127.09, 133.81, 110.56, 84.57, 79.57, 70.72, 65.71, 55.71, 36.56, 31.99, 30.74, 29.64, 29.43, 29.14, 27.04, 12.97. HRMS Calcd. for C₄₅H₅₄Br₃N₃O₁₀S, 1065.1080. Found, 1065.1086.

Electrochemistry Protocols

Cyclic voltammograms were collected in single compartment voltammetric cells equipped with a Au working electrode, Pt wire counter electrode, and Ag/AgNO₃ reference electrode using a CH Instruments 600A electrochemical analyzer as a galvanostat. Prior to use, the Au electrode was thoroughly polished with Al₂O₃ (0.5μ in H₂O) on a felt polishing platform. The electrode was rinsed several times with Milli-Q water and dry methanol immediately before use. The voltammograms were obtained on 3 mL of solutions in specified solvents of 5′-O-(2,2,2-tribromoethoxcarbonyl)-derivative (0.1 mM) and supporting electrolyte (0.01 M). These solutions must be thoroughly dried and degassed prior to reductive scanning at the scan rate of 100 mV/s. Potentials are reported vs. saturated calomel electrode (SCE).

Bulk Electrolysis of 5′-O-(2,2,2-tribromoethoxycarbonyl)-derivatives.

Controlled potential electrolyses were performed in a 3-compartment cell with the compartments separated by a coarse frit. The central compartment contained a magnetic stir bar and the Au foil working electrode (0.025 mm thickness, total area 25 mm×25 mm, actual wetted area 18 mm×18 mm), formed into a half-cylinder. A platinum wire was employed as the anode in another compartment and a Ag/AgNO₃ or saturated calomel reference electrode was used in another compartment. A CH Instruments 600A electrochemical analyzer was used as a potentiostat to maintain a constant pre-set potential difference between the cathode and reference electrode.

Exemplified with 5′-O-(2,2,2-tribromoethoxycarbonyl)-thymidine.

Supporting electrolyte (6 mL of a 0.1 M LiClO₄ solution in dry methanol) was added to the central compartment, and sufficient electrolyte was added to the side compartments to bring them to equal height. Argon was bubbled in all compartments for 30 min to remove dissolved oxygen. The nucleoside derivative (1 mmol) was added to the central compartment. Electrolysis was carried out at −550 mV for 2 h with stirring. The electrolyzed solution was analyzed by HPLC on a reversed-phase column (Econosphere C18 5μ) with a Hewlett Packard 1100 system equipped with a UV-Vis diode array detector. The flow rate was 1 mL/min and used the following gradient: linear from 5% to 20% CH₃CN in 0.05 M ammonium acetate over 15 min, isocratic elution for 10 min, linear from 20% to 5% CH₃CN over 5 min, and isocratic elution for 5 min. The coulombs required for complete reduction: 0.56 C; coulombs observed: 3.6 C.

In the specification, there has been disclosed typical preferred embodiments of the invention and, although specific terms are employed, they are used in a generic and descriptive sense only and not for purposes of limitation of the scope of the invention being set forth in the following claims.

What is claimed is:

1. A method for synthesizing a nucleic acid comprising:
(a) providing a first nucleic acid having a 3′-terminal and a 5′-protecting group;
(b) covalently coupling said 3′-terminal of the first nucleic acid to an electrode; and
(c) cleaving said 5′-protecting group from said first nucleic acid by passing a current therethrough under reaction conditions in which said first nucleic acid remains covalently coupled to said electrode to provide a nucleic acid having a deprotected 5′ terminal.

2. The method according to claim 1, further comprising:
(d) providing a subsequent nucleic acid having a 3′-terminal and a 5′-protecting group;
(e) covalently coupling said 3′-terminal of the subsequent nucleic acid to said deprotected 5′ terminal of said first nucleic acid; and
(f) cleaving said 5′-protecting group from said subsequent nucleic acid by passing a current therethrough under reaction conditions in which said subsequent nucleic acid remains covalently coupled to said deprotected nucleic acid and in which said deprotected nucleic acid remains covalently coupled to said electrode.

3. The method according to claim 2, comprising:
(g) cyclically repeating steps (d) through (f) at least one additional time to produce a further elongated nucleic acid.

4. The method of claim 3, wherein at least one of said first nucleic acid and said subsequent nucleic acid further comprises at least one N-protecting group, said method further comprising the step of:
(h) cleaving the at least one N-protecting group.

5. The method according to claim 1, wherein said covalently coupling is a thiol coupling.

6. The method according to claim 1, wherein said electrode comprises a metal.

7. The method according to claim 6, wherein said metal comprises gold.

8. The method according to claim 1, wherein said electrode comprises carbon.

9. The method according to claim 1, wherein said electrode comprises a metal oxide.

10. An array of nucleic acids comprising:
a microelectronic substrate having at least a first surface;
a plurality of different nucleic acids attached to the first surface of the substrate at a density exceeding at least 1000 different nucleic acids/cm², wherein each of the different nucleic acids is attached to the surface of the microelectronic substrate in a different known location, and has a different determinable sequence;
a plurality of different binding detection electrodes on said first surface; and
wherein each different nucleic acid has a different binding detection electrode operatively associated therewith.

11. The array of claim 10, wherein the plurality of different nucleic acids is attached to the first surface of the substrate by the 3′-terminal of a first nucleic acid.

12. The array of claim 10, further comprising a contact electrically connected to each of said electrodes.

13. The array of claim 10, wherein each nucleic acid is at least two nucleotides in length.

14. The array of claim 10, wherein said electrode comprises a metal.

15. The array of claim 10, wherein said electrode comprises a metal oxide.

16. The array of claim 10, wherein said electrode comprises carbon.

17. The array of claim 10, wherein a second plurality of nucleic acids is covalently coupled to the first plurality of nucleic acids.

18. A method of making an array of nucleic acids comprising:
(a) providing a plurality of nucleotides with a 3′-terminal and a 5′-protecting group;
(b) covalently coupling the 3′-terminal of one of the nucleotides to a first surface of a microelectronic substrate, the nucleotides having different predetermined sequences and being attached at different localized areas having a width of less than 100 microns on the first surface of a microelectronic substrate;
(c) cleaving said 5′-protecting group from the first nucleotide attached to the microelectronic substrate by passing a current therethrough under reaction conditions in which said first nucleotide remains covalently coupled to said electrode to provide elongated oligonucleotides; and
(d) covalently coupling a 3′-terminal of a subsequent nucleotide having a 3′-terminal and a 5′-protecting group to the first nucleotide to produce further elongated oligonucleotides; and
wherein each of said first nucleotide coupled to the microelectronic substrate has a different binding detection electrode operatively associated therewith.

19. The method according to claim 18, further comprising cyclically repeating steps (c) through (d) at least one additional time to produce further elongated oligonucleotides.

20. The method according to claim 18, further comprising deprotecting the 5′-terminal of the terminal nucleotide.

21. The method according to claim 18, further comprising oxidizing the H-phosphonate.

22. The method according to claim 18, wherein said cleaving said 5′-protecting group from said first nucleic acid comprises:
passing a current therethrough under reaction conditions in which said first nucleic acid remains covalently coupled to said microelectronic substrate to provide a deprotected nucleic acid.

23. The method according to claim 18, wherein said electrode comprises a metal.

24. The method according to claim 18, wherein said electrode comprises a metal oxide.

25. The method according to claim 18, wherein said electrode comprises carbon.

26. The method according to claim 18, wherein said covalently coupling is a thiol coupling.
